(19) United States
(12) Patent Application Publication
(10) Pub. No.: US 2025/0256088 A1
(43) Pub. Date: Aug. 14, 2025

(54) Method and Device for the Optimization of Loading a Cardiac Assist Device

(71) Applicant: CorInnova Incorporated, Houston, TX (US)

(72) Inventors: Amie Marie Theall, Houston, TX (US); Barbora Bobakova, Houston, TX (US)

(73) Assignees: Amie Marie Theall, Houseton, TX (US); Barbora Bobakova, Houston, TX (US)

(21) Appl. No.: 18/859,910

(22) PCT Filed: Apr. 27, 2023
(86) PCT No.: PCT/US2023/020130

§ 371 Date: Oct. 24, 2024

Related U.S. Application Data
(60) Provisional application No. 63/335,808, filed on Apr. 28, 2022.

(51) Int. Cl. A61M 60/865; A61M 60/17; A61M 60/187; A61M 60/289; A61M 60/839

(52) U.S. Cl. CPC A61M 60/865; A61M 60/17; A61M 60/187; A61M 60/289; A61M 60/839; A61M 2207/00; A61M 2209/04

(57) ABSTRACT

The present invention provides a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame.

### TECHNICAL FIELD OF THE INVENTION

The present invention relates in general to the field of devices and method to aid in the insertion of a cardiac assist device which are designed to interface with the heart of a patient to improve its pumping function, and, more particularly, to modulate contraction strain patterns on a diseased or damaged heart in order to reduce dyskinetic or hypokinetic motions.

### BACKGROUND ART

Without limiting the scope of the invention, its background is described in connection with methods and devices for delivery or deployment of minimally invasive extra-cardiac devices. Congestive heart failure (CHF) is a condition in which the heart can't pump enough blood to the body's other organs. For people over age 65, it is the number one cause of death, with nearly 290,000 people dying from this disease each year. There are 800,000 people with end-stage CHF and only 2,200 hearts available for transplant each year, leaving a large number of people suffering from this disease. Of that 800,000, about 48,000 are suitable for bridge to transplant and 90,000 patients are suitable for destination therapy. Bridge to transplant refers to the use of ventricle assist devices to sustain severe heart failure patients until a donor heart becomes available and they can receive a transplant. Destination therapy refers to the use of a left ventricular assist device for long-term therapy. It is common for there to be an initial decline in pumping capacity of the heart following heart failure which results in a variety of compensatory mechanisms. The phenomenon of left ventricular remodeling, followed by a change in the wall stress is considered the single most important cause for the worsening of these heart attack patients. Subsequently these patients are rendered helpless and immobile with no options for treatment other than maintenance therapies or placement on a cardiac transplant waiting list.

The various mechanical assist therapies (i.e., drugs, biventricular pacing, blood contacting assist devices, surgical manipulations, or passive stents and constraints etc.) typically off-load the heart and thus only modulate the strain pattern indirectly (e.g., through greater ejection fraction).

One treatment for patients who suffer from either a myocardial infarction or CHF is the implantation of direct cardiac compression device (DCCD). Only DCCDs can directly induce a particular strain pattern. However, most prior DCCDs have been developed for enhancing ejection fraction or for ease of implantation rather than for strain modulation. Currently, a sternotomy is the preferred method of implantation of the DCCD. Sternotomy is a type of surgical procedure in which a vertical inline incision is made along the sternum, after which the sternum itself is divided, or “cracked.” This procedure provides access to the heart for surgical procedures. This procedure is very painful and results in long recovery times with a high risk of infection. Further, there is a high risk of complications due to the lengthy surgery required for these unstable patients.

Current minimally invasive devices may be deployed via a system of guidewires placed between the pericardium and the heart. There is a need to insert minimally invasive assistive biotechnology apparatuses such as a DCCD or a cardiac compression device (CCD) in a minimally invasive way without the use of guidewires.

For example, U.S. Pat. No. 10,398,556, entitled, “Diastolic recoil method and device for treatment of cardiac pathologies,” discloses methods and direct cardiac contact diastolic recoil device to improve diastolic recoil of a heart and includes a biocompatible film attached to or enclosing one or more structural elements that store elastic energy during heart contraction and release energy during heart filling.

U.S. Pat. No. 8,545,387, entitled, “Apparatus and method for minimally invasive implantation of heart assist device,” discloses a method and related apparatus for the minimally invasive implantation about a heart of at least a deployable device. The method comprises the steps of performing a left thoracotomy or subxiphoid incision; obtaining access to the pericardial sac; making a generally linear incision in the pericardial sac; positioning an assembly having an insertion aperture member with an upper ring and a lower ring or flange and insertion tube having therein a deployable device.

U.S. Patent Application Publication No. 20160346449, entitled, “Biomimetic actuation device and system, and methods for controlling a biomimetic actuation device and system,” discloses a biomimetic actuation device includes a flexible substrate, conformable for disposition about an object, defining an apex and a base, bearing at least one soft actuator configured to change state from a first state to a second state upon introduction of a pressurized fluid to an internal volume of the at least one soft actuator.

U.S. Patent Application Publication No. 20160017899, entitled, “Soft actuators and soft actuating devices,” discloses a soft buckling linear actuator is described, including: a plurality of substantially parallel bucklable, elastic structural components each having its longest dimension along a first axis; and a plurality of secondary structural components each disposed between and bridging two adjacent bucklable, elastic structural components; wherein every two adjacent bucklable, elastic structural components and the secondary structural components in-between define a layer comprising a plurality of cells each capable of being connected with a fluid inflation or deflation source; the secondary structural components from two adjacent layers are not aligned along a second axis perpendicular to the first axis; and the secondary structural components are configured not to buckle, the bucklable, elastic structural components are configured to buckle along the second axis to generate a linear force, upon the inflation or deflation of the cells. Methods of actuation using the same are also described.

### DISCLOSURE OF THE INVENTION

The present invention provides a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame. The shaft comprises a shaft diameter of between about 0.25 and 1.0 inches. The film comprises one or more polymers. The tip end comprises a rounded tip. The plethora of wire loops comprises memory metal. The plethora of wire loops comprises nitinol. The plethora of wire loops are attached to the shaft at the tip end. The plethora of wire loops comprises about 12 wire loops, with each of the about 12 loops optionally and at least partially overlapping an adjacent loop. The about twelve wire loops can be 3, 4, 5, 6, 7, 8, 9, 10 11, 15 14, 15, 16 wire loops. The plethora of wire loops may include one or more connections between an adjacent loop.

The present invention provides a method of loading a DCCD into a deployment tube comprising the steps of: providing a DCCD; providing a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame; providing a deployment tube, wherein the deployment tube is connected to a loading funnel; positioning the DCCD around the film; insert the DCCD into the funnel; move the shaft into the funnel such that the DCCD moves into the funnel and compresses the DCCD against the film and compresses the film against the wire frame; insert the DCCD into the deployment tube by inserting the shaft into the deployment tube and compressing the DCCD into the deployment tube and against the film and the wire frame; and removing the DCCD loading tube from the DCCD and the deployment tube. The method further comprises the step of further inserting the DCCD into the deployment tube manually to fully insert the DCCD into the deployment tube. The shaft comprises a shaft diameter of between about 0.25 and 1.0 inches. The film comprises one or more polymers. The tip end comprises a rounded tip. The plethora of wire loops comprises memory metal. The plethora of wire loops are attached to the shaft at the tip end. The plethora of wire loops comprises 12 wire loops with each of the 12 loops at least partially overlapping an adjacent loop. The method further comprises the step of sealing the deployment tube.

The present invention provides a method of inserting a DCCD about a heart in need of treatment comprising the steps of: providing a deployment tube; loading a DCCD into the deployment tube comprising the steps of: providing a DCCD; providing a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame; providing a deployment tube, wherein the deployment tube is connected to a loading funnel; positioning the DCCD around the film; insert the DCCD into the funnel; move the shaft into the funnel such that the DCCD moves into the funnel and compresses the DCCD against the film and compresses the film against the wire frame; insert the DCCD into the deployment tube by inserting the shaft into the deployment tube and compressing the DCCD into the deployment tube and against the film and the wire frame; removing the DCCD loading tube from the DCCD and the deployment tube; providing a patient having the heart in need of treatment; providing access to the heart; inserting the deployment tube through the access and into communication with the heart; ejecting the DCCD to position the DCCD about the heart; withdrawing the deployment tube; and closing the access.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show images of a device deployed around the heart.

FIGS. 2A, 2B and 2C are images of the DCCD compressed into a deployment tube.

FIGS. 3A, 3B, 3C and 3D are images of the loading system components.

FIGS. 4A and 4B are images of the loading tool.

FIG. 5 is an image of the funnel and the deployment tube components.

FIG. 6 is a plot of the loading forces for each prototype.

FIG. 7 is a plot of the average loading time for previous methods versus loading tool method.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention except as outlined in the claims.

As used herein, a “biomedical material” is a material, which is physiologically inert to avoid rejection or other negative inflammatory response.

The present invention is designed to aid in the positioning of a cardiac device about the heart. The present invention may be used to implant a direct cardiac compression device or a cardiac compression device with a collapsible structure that assumes the shape of the left ventricle keeping an inwardly directed pressure to prevent the enlargement of the ventricle and provides adjustment of the end-diastolic volume for limiting/reducing the end-systolic volume.

The present invention provides a tool to aid in the loading of a device for the implantation of a device around the heart in a minimally invasive fashion. The delivery device includes an insertion aperture member that is moved in position into an aperture in the pericardial sac. Within the insertion aperture member is a deployable device, such as a heart assist device that can be extended from the insertion aperture member to encompass the heart. The insertion aperture member can then be removed, leaving the heart assist device positioned about the heart. Alternatively, the insertion aperture member may be secured in the position and remain inserted. In another embodiment, the insertion aperture member May include a first framework that is extended from the insertion aperture member to position the pericardial sac to allow the insertion of a heart assist device. The first framework may be removed once the heart assist device is in position about the heart.

In another embodiment, the present invention provides a tool to aid in the loading of a device into the delivery tube for the delivery device includes an insertion device that is moved in position into an aperture in the pericardial sac. The insertion device includes an insertion tube having a first end aperture separated from a second end aperture by an insertion tube. A second insertion tube is positioned within the first insertion tube. This multi-tube configuration may be constructed using 2 separate and removable insertion tubes or configured such that the first and second insertion tubes are integrated into a single unit. This multi-tube configuration allows a first framework to be inserted in first insertion tube to be used to position the pericardial sac so that the second insertion tube may insert the heart assist device about the heart. Similarly, this may be accomplished by other means, e.g., a single insertion tube having internal channels between the inner and outer walls of the insertion tube. The internal channels can house a frame that can be used to position the pericardial sac.

Within the insertion aperture member is a deployable device, such as a heart assist device that can be extended from the insertion aperture member to encompass the heart. The insertion aperture member can then be removed, leaving the heart assist device positioned about the heart. Alternatively, the insertion aperture member may be secured in the position and remain inserted. In another embodiment, the insertion aperture member may include a first framework that is extended from the insertion aperture member to position the pericardial sac to allow the insertion of a heart assist device. The first framework may be removed once the heart assist device is in position about the heart.

The heart assist device may be any device that is placed about the heart. The device can be a direct cardiac compression device (DCCD) such as the Anstadt cup, as described in U.S. Pat. Nos. 5,119,804, 5,713,954, the heart booster, as described in U.S. Pat. No. 5,713,954, heart harness, mesh electrode, heart support device as described in U.S. Pat. No. 3,983,863 and the direct cardiac compression device (DCCD) of U.S. Pat. No. 10,398,556.

The present invention provides a tool to aid and optimize the insertion of a compression device into the insertion tube so that the implantation of a biomimetic actuation device around the heart is made in a minimally invasive fashion. The delivery device includes an insertion aperture member that is moved in position into an aperture in the pericardial sac, within the insertion aperture member is a deployable biomimetic actuation device that can be extended from the insertion aperture member to encompass the heart. The insertion aperture member can then be removed leaving the heart assist device positioned about the heart. Other heart assist devices include the devices seen in U.S. Pat. Nos. 7,445,593, 7,935,045, 8,187,160, 8,944,986, 9,510,746, 10,398,556 and U.S. Patent Application Publication Nos. 2011/0021864, 2004/0167375, 2006/0142634, and 2016/0346449 and similar patents and patent applications.

A deployable device is implanted in a heart in accordance with the method of the present invention, using the assembly hereinabove described, as follows. A normal left thoracotomy or subxiphoid incision is performed to allow access.

The present invention provides a mechanism to insert the heart assist device around the heart in a minimally invasive manner. This is accomplished by an insertion means that allows the assist device to be inserted into the delivery device and pushed through the insertion device.

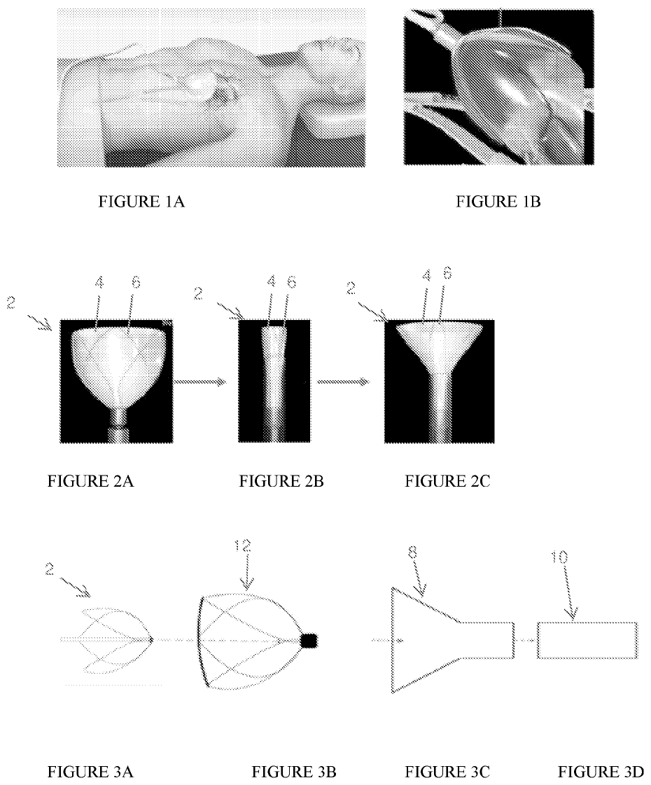

FIGS. 1A and 1B show images of a device deployed around the heart. The present invention provides a tool to aid in the insertion of the device into the delivery tube prior to being deployed around the heart. The device is deployed through a small incision in the apex of the pericardium to form around the ventricles within the pericardial sac. The present invention provides a loading method that has a high impact on successful deployment; where a failed deployment can result in bodily harm or unsuccessful placement.

FIGS. 2A, 2B, and 2C are images of the cardiac compression device compressed into the deployment tube. The device 2 includes a delicate thin-film polyurethane 4 and super elastic shape memory Nitinol wire 6. Although any suitable material may be used as the film and the shape memory wire. The film may be any suitable polymer, a combination of polymers, or layers of polymers known to the skilled artisan. The film may also be of the same composition throughout the film or may be a composite of different materials. This composite may be in the form of layers of different materials, separate regions of the polymer may have different compositions to account for specific properties at specific regions of the film. For example, the center of the film may be reinforced with an addition layer of polymer to spread out the strain on that portion of the film. Similarly, the film may include fibers, nanostructures, substructures, or other components that are used to provide specific properties to the film. For example, vertical fibers may be used to provide strength to the film in the vertical direction. These materials allow it to be compressed into an approximately 1-inch diameter tube for a minimally invasive deployment and implantation; however, the tube diameter may be between about 0.25 inches and about 1.5 inches, depending on the specific application. As used herein, the term about specifically refers to a variation of up to 15 to 20% (±15 to 20%). In contrast, the current loading method involves manually squeezing the DCCD to compress and load the DCCD into the deployment tube.

FIGS. 3A, 3B, 3C, and 3D are images of the loading system components. The loading system consists of three primary components: the loading tool 2, funnel 8, and deployment tube 10. The loading tool is a significant component. The loading tool is used to navigate the DCCD 12 through a funnel into the deployment tube. The loading tool may be made of a polymer material, metal, composite, hybrid material, or other suitable material for use in the system. The loading tool may include a handle that extends and terminates at a tip. The tip may have a blunted, rounded, concave, convex, squared, oval, or free formed shape. In addition, the tip may include a recess that fits an insert that forms an interface. In some embodiments, the insert may be compressible to aid in the interface. In one embodiment, the loading tool is made of a polycarbonate handle that allows insertion into the DCCD and forces the application to guide DCCD and the nitinol frame mimics the DCCD shape and is used to hold the carbothane layer in place. Although, this embodiment uses a carbothane layer other aliphatic and aromatic, polycarbonate-based thermoplastic polyurethanes and even other polymers may be used. The frame is covered with a polypropylene (PP) cone to prevent carbothane pinching. The cover may be made from a polymer material, metal or other suitable material. The loading funnel provides a conical shape which supports the outer wall of the DCCD slowly compressing and guiding it into the deployment tube. In addition, the funnel is temporarily connected to the deployment tube with an adapter. The adaptor may be made from rubber, polymer, elastomer, or other suitable material. The deployment tube is the device in which the device is loaded to and deployed from. The deployment tube is used to retain the DCCD or other device to be deployed around the heart. The deployment tube retains the device in a Polytetrafluoroethylene (PTFE) Tube design for its low friction properties, although other tetrafluoroethylenes and other polymers may be used.

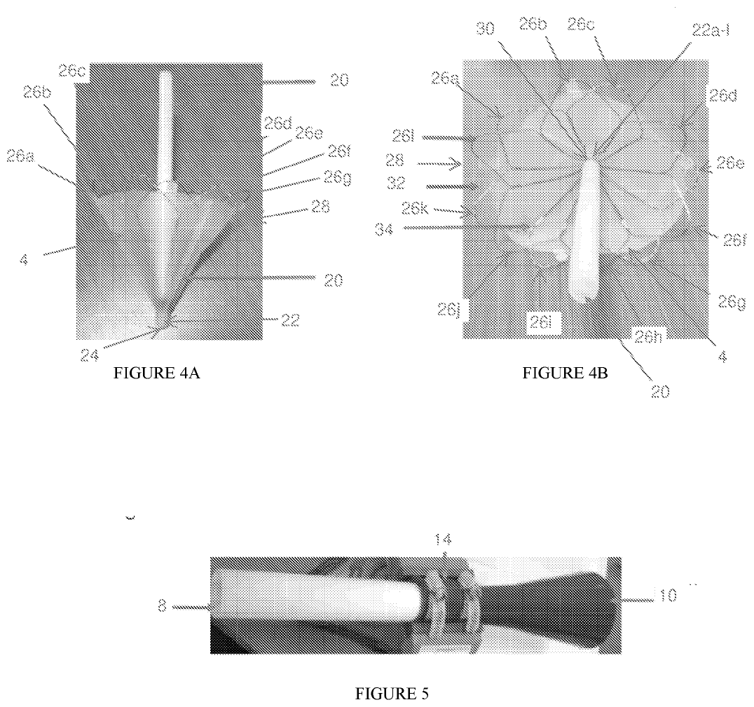

FIGS. 4A and 4B are images of the loading tool. The loading tool 2 includes a handle 20. The handle 20 may be machined, molded, 3D printed formed and may be made from a PC to form a rod with a rounded end 22. In some embodiments, the handle 20 forms a smooth shape to reduce the chance of interfering with or puncturing the device. Although the rounded end 22 may have any end structure that is necessary. The device includes twelve wires 26a-l that are structured with multiple bends to form a nitinol wire frame 28. Twelve holes 30 are placed near the rounded end tip 24 for the placement of Nitinol wire ends of the Nitinol frame 28. The loading tool 2 may include a nitinol wire frame 28. The twelve compressible Nitinol wires 26a-l bent in the shape of the nitinol frame of the DCCD. Free wire ends are placed into the respective holes 30 on the handle 20. The loading tool 2 includes a suture mesh 32. The suture mesh 32 reinforces links between Nitinol wires 26a-l to provide stability. The loading tool includes crimps 34 placed on the lower bend of Nitinol wires 26a-l to link them together. The loading tool includes a PP cone 4 surrounding the Nitinol wires 26a-l to protect the containment layer of DCCD from damage. Although the embodiment shows twelve Nitinol wires 26a-l the device may have 1, 2, 3, 4, 5, 6, 7, 8, 9, 10, 11, 13, 14, 15, 16, 17, 18, 19, 20 21, 22, 23, 24, 25, 26, 27 28 or more wires depending on the specific configuration. In some configurations, the device may include a single wire that is positioned to form numerous loops to have the same structure as shown with twelve individual wires positioned to form loops. Similarly, fewer wires may be used to simulate numerous independent wires.

FIG. 5 is an image of the funnel 8 and the deployment tube 10 components. FIG. 5 shows a clamp 14 connector that secures a funnel 8 and a deployment tube 10 together for DCCD loading. Once loading is complete, components are disconnected and the DCCD is ready for surgical implantation.

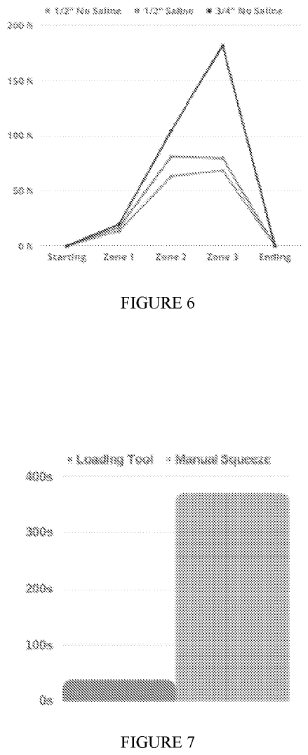

FIG. 6 is a plot of the loading forces for each prototype. The three samples include a ½ inch with no saline (purple), ½ inch with saline (red) and ¾ inch with no saline (brown). The loading path was divided into 3 zones and the maximum force was measured for each zone. Loading forces decreased with decreasing tool handle diameter and were lower without the use of saline. The lowest forces, or easiest loading, was with a handle of ½″ diameter and no saline.

FIG. 7 is a plot of the average loading time for previous methods versus the present method of loading using the loading tool. The red bar indicates the average loading time using the loading tool, whereas the pink bar indicates the average time for loading manually. The graph compares the average loading time for previous methods versus the loading tool method. The average loading time decreased by 85.7% when using the newly developed loading tool method compared to previous manual squeeze methods.

Tables 1 below is a carbothane layer leak testing. Following the testing of loading forces for each loading tool variation, the DCCD's outer carbothane layer was checked for leaks. The plastic was filled with air, placed in a water bath, and inspected for the formation of bubbles. Observed bubbles indicate leaking and a failed test. Only the tool with the ¾ inch handle failed.

TABLE 1
Containment Leak Test

| | |
|---|---|
| d = ½″ Saline | PASS |
| d = ½″ No Saline | PASS |
| d = ¾″ No Saline | FAIL |

The present invention provides a loading tool system that is able to successfully navigate the DCCD into the deployment tube while significantly reducing the loading time. Required loading force is minimized by a reduction in tool handle diameter. As a result, the DCCD is properly packed in the loading tube, allowing it to unfold in the desired orientation. Future works may include reducing the loading handle diameter while maintaining its durability. Furthermore, there is possibility for further improvement with the deployment tube and funnel component geometries.

Other suitable polymers include elastomeric polyurethane, latex, polyetherurethane, polycarbonateurethane, silicone, polysiloxaneurethane, hydrogenated polystyrene-butadiene copolymer, ethylene-propylene and dicyclopentadiene terpolymer, hydrogenated poly(styrene-butadiene) copolymer, poly(tetramethylene-ether glycol) urethanes, poly(hexamethylenecarbonate-ethylenecarbonate glycol) urethanes and combinations thereof.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A DCCD loading device comprising:
a shaft that extends from a tip end to a handle;
a wire frame in contact with or adjacent to the tip end, wherein the wire frame comprises a plethora of wire loops extending away from the tip; and
a film in contact with the tip end and extending over at least a portion of the wire frame.

2. A method of loading a DCCD into a deployment tube comprising the steps of:
providing a DCCD;
providing a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame;
providing a deployment tube, wherein the deployment tube is connected to a loading funnel;
positioning the DCCD around the film;
insert the DCCD into the funnel;
move the shaft into the funnel such that the DCCD moves into the funnel and compresses the DCCD against the film and compresses the film against the wire frame;
insert the DCCD into the deployment tube by inserting the shaft into the deployment tube and compressing the DCCD into the deployment tube and against the film and the wire frame; and
removing the DCCD loading tube from the DCCD and the deployment tube.

3. The method of claim 10, further comprising the step of further inserting the DCCD into the deployment tube manually to fully insert the DCCD into the deployment tube.

4. The DCCD loading device of claims 1-3, wherein the shaft comprises a shaft diameter of between about 0.5 and 1.0 inches.

5. The DCCD loading device of claims 1-4, wherein the film comprises one or more polymers, one or more layers of polymer or a combination thereof.

6. The DCCD loading device of claims 1-5, wherein the tip end comprises a rounded tip, a blunted tip, a convex tip, a concave tip.

7. The DCCD loading device of claims 1-6, wherein the plethora of wire loops comprises one or more metals, one or more polymers or a combination thereof.

8. The DCCD loading device of claims 1-7, wherein the plethora of wire loops comprises memory metal, wherein the plethora of wire loops preferably comprises nitinol.

9. The DCCD loading device of claims 1-8, wherein the plethora of wire loops comprises about 12 wire loops with each of the about 12 loops at least partially overlapping an adjacent loop.

10. The DCCD loading device of claims 1-9, wherein the plethora of wire loops comprises one or more connections between an adjacent loop.

11. A method of inserting a DCCD about a heart in need of treatment comprising the steps of:
providing a deployment tube;
loading a DCCD into the deployment tube comprising the steps of: providing a DCCD; providing a DCCD loading device comprising: a shaft that extends from a tip end to a handle; a wire frame in contact with the tip end, wherein the wire frame comprises a plethora of wire loops extending away from the tip; and a film in contact with the tip end and extending over at least a portion of the wire frame; providing a deployment tube, wherein the deployment tube is connected to a loading funnel; positioning the DCCD around the film; insert the DCCD into the funnel; move the shaft into the funnel such that the DCCD moves into the funnel and compresses the DCCD against the film and compresses the film against the wire frame; insert the DCCD into the deployment tube by inserting the shaft into the deployment tube and compressing the DCCD into the deployment tube and against the film and the wire frame; removing the DCCD loading tube from the DCCD and the deployment tube;
providing a patient having the heart in need of treatment;
providing access to the heart;
inserting the deployment tube through the access and into communication with the heart;
ejecting the DCCD to position the DCCD about the heart;
withdrawing the deployment tube; and
closing the access.
